(19) United States
(12) Patent Application Publication
(10) Pub. No.: US 2013/0053861 A1
(43) Pub. Date: Feb. 28, 2013

(54) OPEN SURGICAL PROSTHESIS DEPLOYMENT SYSTEM

(71) Applicants: Donald F. Patterson, Bloomington, IN (US); Laura A. Boehm, Hamilton, OH (US); Morgan K. T. Humphrey, Owensboro, KY (US); Jeremy E. Phillips, Glasgow, KY (US); Sally A. Zimmerman, Ft. Thomas, KY (US); Amro Kamel, Bloomington, IN (US)

(72) Inventors: Donald F. Patterson, Bloomington, IN (US); Laura A. Boehm, Hamilton, OH (US); Morgan K. T. Humphrey, Owensboro, KY (US); Jeremy E. Phillips, Glasgow, KY (US); Sally A. Zimmerman, Ft. Thomas, KY (US); Amro Kamel, Bloomington, IN (US)

(73) Assignee: Cook Medical Technologies LLC, Bloomington, IN (US)

(21) Appl. No.: 13/218,866

(22) Filed: Aug. 26, 2011

(51) Int. Cl. A61B 17/00

(52) U.S. Cl. 606/108

(57) ABSTRACT

A device is used for deploying a prosthesis to vessel portions of a body vessel. The device can include a retaining member and a sheath. The retaining member, such as clamps, is movable between closed and open positions. In the closed position, the retaining member compresses an intermediate segment of the prosthesis. In the open position, the retaining member is positioned away from the prosthesis to allow for expansion of the intermediate segment. The sheath compresses an outer segment of the prosthesis, where the outer segment is associated with a first or second outer end of the prosthesis. The sheath is removable from the outer segment to allow for expansion thereof when the retaining member is in the closed position. An actuation member is operable to move the retaining member between the closed and open positions. Clamps are pivotable in response to movement of the actuation member.

### BACKGROUND

The present disclosure relates generally to medical devices for emergency open surgical repair of body structures that define a lumen. More particularly, it relates to deployment systems used for repairing damaged body structure and gaining fluid stability during emergency open surgical medical procedures.

Trauma physicians frequently encounter patients having traumatic injury to a body vessel, such as lacerated vessels or even transected vessels, resulting from gunshots, knife wounds, motor vehicle accidents, explosions, etc. Significant damage to a body vessel may expose a patient to deleterious conditions such as the loss of a limb, loss of function of a limb, increased risk of stroke, impairment of neurological functions, and compartment syndrome, among others. Particularly severe cases of vascular injury and blood loss may even result in death. In such severe situations, the immediate goal is to obtain hemostasis or fluid stability while maintaining perfusion of adequate blood flow or fluid flow to critical organs, such as the brain, liver, kidneys, and heart.

Examples of treatment that are commonly performed by trauma physicians to treat body vessel injuries include the clamping of the vessel with a hemostat, the use of a balloon tamponade, the ligation of the damaged vessel at or near the site of injury, or the insertion of one or more temporary shunts. However, conventional surgical repair is generally difficult with such actively bleeding, moribund patients. In many instances, there is simply not enough time to repair the body vessel adequately by re-approximating and suturing the body vessel. In many situations, the trauma physician will simply insert a temporary shunt (such as a Pruitt-Inahara Shunt) into the vessel. However, use of temporary shunts has been linked to the formation of clots. This may require returning the patient to the operating room for treatment and removal of the clots, often within about 36 to 48 hours of the original repair. Since shunts are generally placed as a temporary measure to restore blood flow and stop excessive blood loss, the shunt is typically removed when the patient has stabilized (generally a few days later) by a specialized vascular surgeon. After removal, the vascular surgeon will replace the shunt with a vascular graft, such as a fabric graft that is sewn into place. With respect to ligation, ligation of the damaged blood vessel may result in muscle necrosis, loss of muscle function, or a potential limb loss or death.

Due to the nature of the body vessel injury that may be encountered, the insertion of shunts or ligation of a blood vessel, for example, often requires that such treatments be rapidly performed at great speed, and with a high degree of physician skill. Such treatments may occupy an undue amount of time and attention of the trauma physician at a time when other pressing issues regarding the patient's treatment require immediate attention. In addition, the level of particularized skill required to address a vascular trauma may exceed that possessed by the typical trauma physician. In particular, traumatic episodes to the vessel may require the skills of a physician specially trained to address the particular vascular trauma, and to stabilize the patient in the best manner possible under the circumstances of the case.

Some open surgical techniques utilize sutures to affix damaged tissue portions surrounding fittings that have been deployed with the vessel, which requires the trauma physician to take time to tie the sutures properly. Although in modern medicine sutures can be tied in relatively rapid fashion, any step in a repair process that occupies physician time in an emergency situation is potentially problematic. In addition, the use of sutures to affix the vessel to the fitting compresses the tissue of the vessel against the fitting. Compression of tissue may increase the risk of necrosis of the portion of the vessel tissue on the side of the suture remote from the blood supply. When present, necrosis of this portion of the vessel tissue may result in the tissue separating at the point of the sutures. In this event, the connection between the vessel and the fitting may eventually become weakened and subject to failure. If the connection fails, the device may disengage from the vessel. Therefore, efforts continue to develop techniques that reduce the physician time required for such techniques, so that this time can be spent on other potentially life-saving measures, and the blood flow is more quickly restored and damage caused by lack of blood flow is minimized.

Trauma physicians generally find it difficult to manipulate a prosthesis for insertion into a body vessel that has been traumatically injured. For example, one difficulty arises from the trauma physician trying to limit the size of the opening created for gaining access to the injured vessel so that such opening requiring healing is as small as possible. Another difficulty is that the injured vessel can be anywhere in the body, having different surrounding environments of bone structure, muscle tissue, blood vessels, and the like, which makes such obstructions difficult to predict in every situation and leaves the trauma physician working with an even further limited access opening. Another potential consideration is the amount of body vessel removed during a transection. The goal would be to remove a portion of the body vessel as small as possible. Yet, a small portion removed from the vessel leaves such a small space between the two vessel portions, thereby making it difficult to introduce the prosthesis between the two vessel portions.

Thus, what is needed is a deployment device for delivering a prosthesis for use in repair of an injured body vessel, such as an artery or a vein, (and in particular a transected vessel) during emergency surgery. It would be desirable if such deployment device was easy for a trauma physician to use, and can rapidly introduce a prosthesis into two vessel portions of a transected vessel, thereby providing a conduit for blood within the injured body vessel.

### SUMMARY

Accordingly, a deployment device is provided herein to address at least some of the shortcomings of the prior art. The device can include a retaining member and a sheath portion. The retaining member is movable between a closed position and an open position. In the closed position, the retaining member can be positioned over at least an intermediate segment of a prosthesis to retain the segment in a compressed configuration, whereas in the open position, the retaining member can be positioned away from the prosthesis to allow the segment to move to an expanded configuration. The sheath can be configured to retain at least an outer segment of the prosthesis in a compressed configuration. The outer segment can be associated with at least one of a first outer end and a second outer end of the prosthesis. The sheath is removable from the outer segment to allow for expansion thereof when the retaining member is in the closed position. An actuation member may be coupled to the retaining member, and is movable between a first position and a second position to move the retaining member between the closed and open positions. A support frame may be provided to house the actuation member. The retaining member may have a first clamp and a second clamp pivotably coupled to the support frame. In one aspect, the first and second clamps can be slidably engaged with a distal portion of the actuation member such that linear movement of the actuation member between the first and second positions causes the clamps to pivot between the open and closed configurations.

In another embodiment, a deployment system can include a prosthesis, a retaining member, and an actuation member. The prosthesis is radially movable between a compressed configuration and an expanded configuration, the prosthesis oriented about a longitudinal axis. The retaining member is movable between a closed position and an open position. In the closed position, the retaining member can be positioned over an intermediate segment of the prosthesis to retain the intermediate segment in the compressed configuration. In the open position, the retaining member can be positioned away from the prosthesis to allow the intermediate segment to move to the expanded configuration. The actuation member can be coupled to the retaining member and operable to move the retaining member between the closed and open positions. The actuation member is movable along a translational axis between a first position and a second position to correspond to the closed and open positions of the retaining member.

In yet another embodiment, a method of treating a body vessel can include one or more of the following steps. A first outer end of a prosthesis can be inserted in a first vessel portion of a body vessel. The prosthesis may have an intermediate segment retained in a compressed configuration with a retaining member. A second outer end of the prosthesis can be inserted in a second vessel portion of the body vessel. The retaining member can be removed from the intermediate segment of the prosthesis, whereby the intermediate segment and the first and second outer ends of the prosthesis are allowed to move to the expanded configuration. In one aspect, the first and second outer ends of the prosthesis can be retained in the compressed configuration with a retaining sheath. The retaining sheath can be removed from the first and second outer ends after being inserted to allow for expansion and engagement between the first and second outer ends of the prosthesis and the corresponding first and second vessel portions prior to removing the retaining member. In another aspect, the intermediate segment of the prosthesis can be retained in the compressed configuration by a first clamp and a second clamp. An actuation member can be coupled to the first and second clamps and is operable to cause movement of the clamps away from intermediate segment to allow for expansion of the intermediate segment of the prosthesis.

### BRIEF DESCRIPTIONS OF THE DRAWINGS

FIG. 1 is a perspective view of an example deployment device pre-loaded with a prosthesis having portions retained by a removable sheath.

FIG. 2 is an exploded perspective view of components that form the deployment device of FIG. 1.

FIG. 3 is a front view of a distal end of the deployment device of FIG. 1 with the pre-loaded prosthesis.

FIG. 4 is a perspective view of a pair of clamps that define an example retaining member of a deployment device in a closed configuration.

FIGS. 5A-5B are perspective views of one of the clamps in FIG. 4.

FIG. 6 is a perspective view of the deployment device of FIG. 1, depicting release of the prosthesis from the device.

FIG. 7 is a perspective view of a distal end of the deployment device of FIG. 1, with the removable sheath removed from outer ends of the prosthesis.

FIGS. 8A-8B are perspective views of another example deployment device pre-loaded with a prosthesis having portions retained by a removable sheath.

FIGS. 9A-9F illustrate a method of deploying a prosthesis with an example deployment device.

### DETAILED DESCRIPTION OF THE DRAWINGS AND THE PRESENTLY PREFERRED EMBODIMENTS

For the purposes of promoting an understanding of the principles of the present disclosure, reference will now be made to the embodiments illustrated in the drawings, and specific language will be used to describe the same. Throughout the specification, when referring to a medical device, or a portion of a medical device, the terms “distal” and “distally” shall denote a position, direction, or orientation that is generally towards, or in the direction of, the patient when the device is in use. The terms “proximal” and “proximally” shall denote a position, direction, or orientation that is generally away from the patient, or closer to the operator, during use of the device. It is understood that like-referenced numerals are used throughout the Figures to designate similar components.

The deployment device described herein can be useful for repair of vessels, lumens, ducts, or passageways of the body, with the term “body vessel” used in the specification to describe theses structures in general, during emergency open surgical repair. This device can be particularly useful for introducing a prosthesis for repair of a lacerated artery or vein during emergency surgery, and particularly, to obtain fluid stability or hemostasis while maintaining blood perfusion. For instance, during an open surgical procedure a trauma pathway is created in the body by the physician to access the desired body vessel. The trauma pathway is typically perpendicular or obliquely angled relative to the desired body vessel. To this end, the deployment device provides a mechanism to deploy the prosthesis within the body vessel portions, while maintaining the device within the trauma pathway and relatively perpendicular to the desired body vessel. In one example, the deployment device can include a retaining member and a removable sheath. The retaining member can move between closed and open positions. In the closed position, the retaining member is placed over an intermediate segment of the prosthesis to retain the intermediate segment in a compressed configuration. In the open position, the retaining member is positioned away from the prosthesis to allow for expansion of the intermediate segment. The removable sheath can retain one or both outer segments of the prosthesis in a compressed configuration. The outer segment can be associated with a first and/or second outer end of the prosthesis. The removable sheath can be removed from the outer segment to allow for expansion of the outer segment of the prosthesis when the retaining member is in the closed position. Preferably, expansion of the outer segment is after the outer segment of the prosthesis is inserted into the respective vessel portions. An actuation member is operable to move the retaining member between the closed and open positions. Clamps can define the retaining member and can be pivotable in response to linear movement of the actuation member. Other applications for the deployment device will become readily apparent to one skilled in the art from the detailed description.

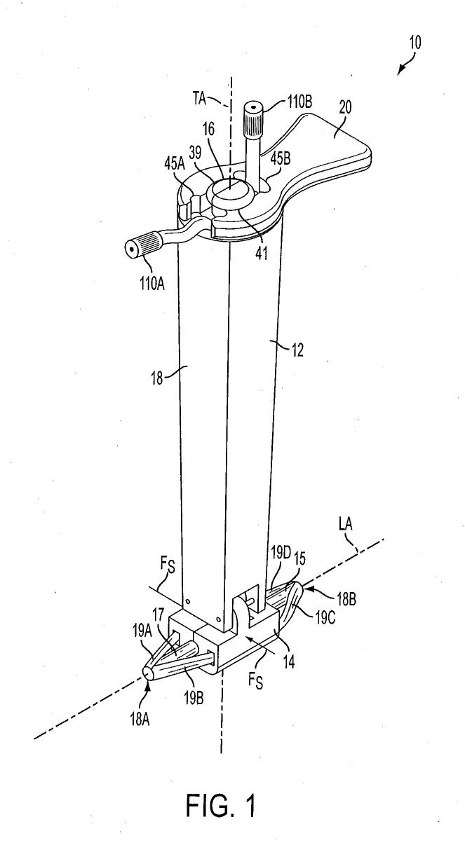

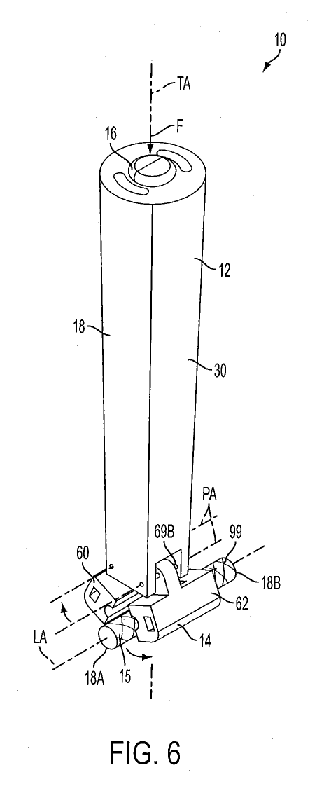

FIG. 1 depicts an example deployment device 10 having a handle portion 12 and a retaining member 14 that can retain at least a partial length of a prosthesis 15 in a radially compressed configuration. The retaining member 14 is movable between a closed position, whereby a prosthesis segment is in the radially compressed configuration, and an open position, whereby the prosthesis segment is permitted to move to a radially expanded configuration. The handle portion 12 can include an actuation member 16 coupled to a support frame 18. The actuation member 16 is configured to move relative to the support frame 18 between a first position and a second position. When the actuation member 16 is in the first position as shown in FIG. 1, the retaining member 14 is in the closed position. When the actuation member 16 is in the second position, the retaining member 14 is in the open position, as shown in FIG. 6.

Some portions of the prosthesis 15 may also be retained by a removable tubular sheath 17, although the deployment devices described herein are operable without the use of the removable sheath. The removable sheath 17 may be fitted at least partially over the first and second outer ends 18A, 18B of the prosthesis 15 to selectively retain the outer ends in the compressed configuration for insertion into an end opening of the a vessel. In one example, the removable sheath may retain the entire prosthesis from the first end to the second end of the prosthesis. In another example, the removable sheath may include two sheath portions, with a first sheath portion retaining one of the outer segments of the prosthesis 15 associated with the first outer end 18A, and a second sheath portion retaining the other outer segment of the prosthesis 15 associated with the second outer end 18B. In other words, the removable sheath may not be disposed over the intermediate portion, which is retained instead by the retaining member 14. One or more retraction members, such as retraction members 19A, 19B, 19C, 19D, can be coupled to the removable sheath 17. The retraction member is operable for peeling and/or splitting the removable sheath from the respective outer ends 18A, 18B toward the middle of the prosthesis 15 to allow for expansion thereof during or after the retaining member 14 is moved from the closed position. To this end, the retraction member may have a slackness to allow for a segment of the retraction member to be radially oriented for insertion into the body vessel along with the outer end of the prosthesis. In one example, the retraction member and the removable sheath are formed from single piece of sheath material. Here, the outer end of the removable sheath is everted along the prosthesis to form the retraction member. Alternatively, the retraction member may include a tension member, such as a wire or a filament, metal or plastic, having a first end attached to the outer end of the removable sheath and a second end accessible outside the body.

The removable sheath 17 may be configured for peeling and/or splitting by use of any well-known means or material that permits the tubular sheath to be separated, preferably longitudinally, along a relatively predictable path. The removable sheath 17 may be pre-split at the base of the retraction members. The removable sheath is usually, but not necessarily separated into two or more portions, thereby opening a fissure along the length that permits its removal from around the prosthesis situated therein. A predetermined split line may be formed in the sheath through which the tear or split progresses due to properties of, and/or features incorporated into the sheath material. Preferably, the mechanism for splitting the removable sheath can withstand being subjected to a curve to the degree required by the particular application without kinking or premature separation. In one example, the removable sheath can comprise a splittable polymer such as molecularly oriented, non-isotropic PTFE that is used to make the PEEL-AWAY® Introducer Sheath. Such sheath is commercially available from Cook Medical Inc. (Bloomington, Ind.), which is also described in, e.g., U.S. Pat. No. 4,306,562 to Osborne and U.S. Pat. No. 4,581,025 to Timmermans, each of which is incorporated herein by reference in its entirety. In another example, the split line can be enhanced by adding at least one preweakened feature, such as a score line, perforations, or reduced wall thickness regions, extending longitudinally along the length of the removable sheath. The longitudinal preweakened feature may be included anywhere from one or more orthogonal predetermined split lines to a helical type arrangement that may comprise only a single predetermined split line. The preweakened feature may have sheath portions that engage each other with a zipper-like or tongue-and-groove-like interface, or any other splittable connection interface along the contacting lateral edges of the sheath portion. Other examples of removable sheath configurations can be found in U.S. Pat. No. 6,447,540 to Fontaine et al. and U.S. Pat. No. 6,827,731, each of which is incorporated herein by reference in its entirety. The removable sheath can have more than one split lines.

A safety lock 20 can be removably attached to the actuation member 16 to maintain the actuation member 16 in the first position and prevent movement of the retaining member 14 to the open position, thereby preventing inadvertent expansion of the prosthesis. Removal of the safety lock 20 from engagement with the actuation member 16 can permit the actuation member to move the second position.

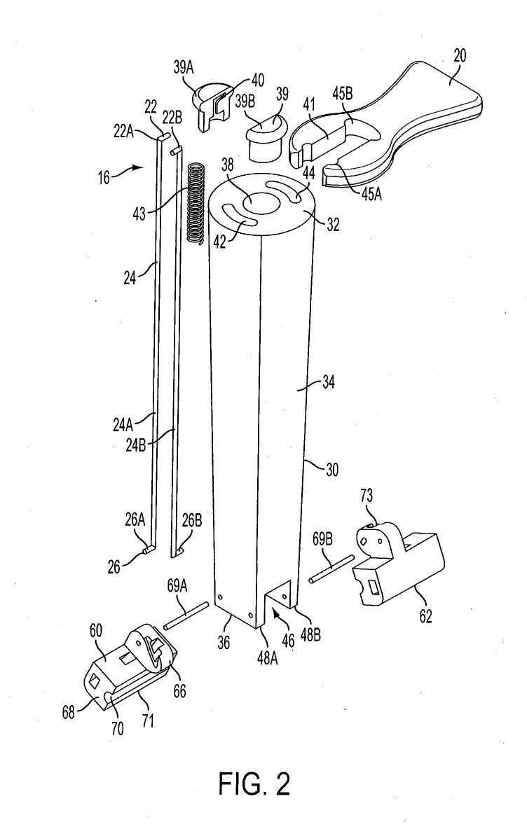

According to FIG. 2, the actuation member 16 can include a first or proximal portion 22, an intermediate body 24, and a second or distal portion 26 coupled to one another. The support frame 18 can include a tubular body 30 that can have a proximal end 32, an intermediate portion 34, and a distal portion 36 coupled to one another. The tubular body 30 can define a passageway 38 about a translational axis TA extending through the tubular body. The translational axis TA can be substantially perpendicular to a longitudinal axis LA of the prosthesis 15 when loaded into the retainer member 14, as shown in FIG. 1. The passageway 38 is sized to allow the intermediate body 24 of the actuation member 16 to pass therethrough so that the actuation member 16 is capable of being translated along the translation axis TA between the first and second positions. The passageway 38 can include additional features, such as bearings and/or lubrication, to facilitate translation of the actuation member.

The proximal portion 22 of the actuation member 24 can extend proximally beyond the proximal end 32 of the tubular body 30. A button 39 can be coupled to the proximal portion 22 to increase the surface area of contact for the end user during operation of the actuation member 16. The button 39 can be an enlarged member having a cross-section that is larger than the cross-section of the proximal end of the passageway 38. The proximal surface of the button 39 can be planar and may be further frictionally enhanced with surface irregularities, such as grooves, dimples, protrusions, or the like, formed in the proximal surface to improve grippability for the end user. The distal portion 26 of the actuation member can extend distally beyond the distal end of the passageway 38 of the support frame. The distal portion 26 can be coupled to the retainer member 14 in a manner to move the retainer member between the closed and open positions.

The intermediate body 24 is configured to transfer forces from the proximal portion 22 of the actuation member to the distal portion 26 of the actuation member. To this end, the intermediate body 24 can be a rigid elongated body that generally can extend substantially parallel to the translation axis TA. In one example, the intermediate body is substantially linear, i.e., does not have bent portions at the axial ends. In another example, the axial ends of the intermediate body can be enlarged or bent to increase surface area contact. For instance, the proximal portion 22 of the intermediate body 24 can be a rigid elongated body, which may extend obliquely, or at least perpendicular, to the translation axis TA. The proximal portion 22 can be fixed in a secured relationship to the intermediate body 24 by any attachment mechanism, such as by adhesives, soldering, welding, or other common attachment mechanisms. The distal portion 26 of the actuation member can be a rigid elongated body that can generally extend obliquely, or at least perpendicular, to the translation axis TA. In one example, the proximal portion 22 and the distal portion 26 extend in opposite directions away from the translational axis TA, or may extend in the same direction.

In one example, the actuation member 16 can include multi-components, although the actuation member can be formed from a single piece. The intermediate body 24 can include two shafts 24A, 24B. The proximal portion 22 and distal portion 26 can be formed by bent regions 22A, 22B, 26A, 26B along the respective ends of each shaft. The button 39 can be formed from two halves 39A, 39B, which when coupled form a disc shape with a smaller diameter portion and an outer flange, as shown in FIG. 1. A recess 40 can be formed in each of the halves 39A, 39B that is sized and shaped, such as, e.g., L-shaped, to receive the respective proximal portion 22A, 22B of the corresponding shaft 24A, 24B. The recess 40 can have an end opening extending from the distal surface of the button half 39A, 39B and can be accessible from a confronting lateral surface of each of the halves 39A, 39B. The button halves 39A, 39B can be attached to the respective proximal portions 22A, 22B by any attachment mechanism, such as by mechanical interference fit, adhesives, soldering, welding, or other common attachment mechanisms. The safety lock 20 includes edges 41 that can fit around the smaller diameter portion of the button and underneath the outer flange of the button to maintain the button and thus the actuation member at the first position.

One or more biasing members 43 can be coupled between the support frame 18 and the actuation member 16 to bias the actuation member in either the first or the second position, preferably in the first position, and thus the retainer member 14 in either the open or the closed positions, preferably in the closed position. In one example, the biasing member 43 is a compression spring having a first end engaged with the distal end of the button, such as an aperture receiving the first end of the compression spring. The compression spring can be extended within the passageway 39 so that the second end of the compression spring is engaged with an inner radial flange (not shown) formed in the passageway. The compression spring may surround the intermediate body 24, such as the shafts 24A, 24B. The biasing member 43 can provide an axial spring force to the proximal portion 22, which in turns provides the retaining member 14 a radially inward retaining force Fs that is substantially perpendicular to the translational axis TA. The combined opposed radially inward retaining forces are greater than the radial expansion forces of the prosthesis such that the clamps can retain the prosthesis in the compressed configuration.

One or more channels, such as channels 42, 44, can be formed into the tubular body 30 along side of the passageway 38 to permit the retraction members to pass therethrough to the proximal end of the device 10. The cross-sectional shape of the channels can be any shape known in the art, such as, e.g., circular, rectangular, elliptical, or oblong (as shown). The channels 42, 44 can be positioned on opposite sides of the passageway to correspond to the outer ends of the prosthesis. A pair of openings 45A, 45B can be formed in the safety lock 20 to allow for the retraction members to pass through from the channels 42, 44.

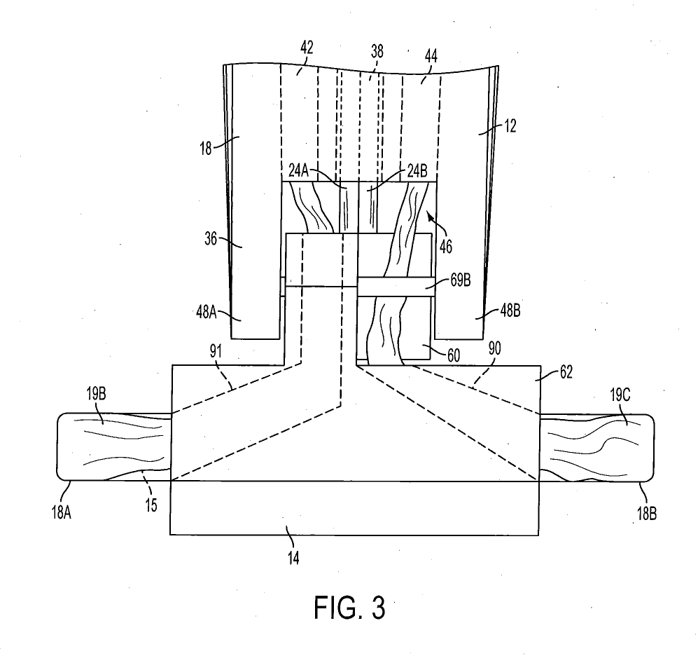

In FIG. 3, the distal portion 36 of the tubular body 18 can have a variety of geometric shapes. For example, a notch 46 can be formed in the distal portion 36. In one example, the notch 46 has a rectangular shape extending between opposite lateral surfaces of the tubular body. The notch 46 can extend perpendicular to the translation axis TA and to the longitudinal axis LA. The notch 46 can be formed in an intermediate portion such that a pair of legs 48A, 48B depends from an inner surface of the notch 46. The legs 48A, 48B can form part of a hinge segment with the retaining member.

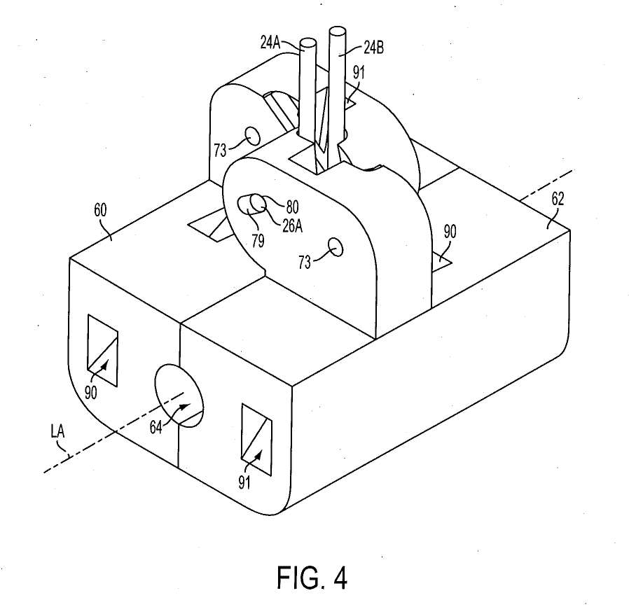

FIGS. 2 and 4 depict one example of the retaining member 14 being formed from a first clamp 60 and a second clamp 62. The clamps 60, 62 together can define a prosthesis retaining chamber 64 about the longitudinal axis LA of the prosthesis 15 when the retaining member is in the closed position.

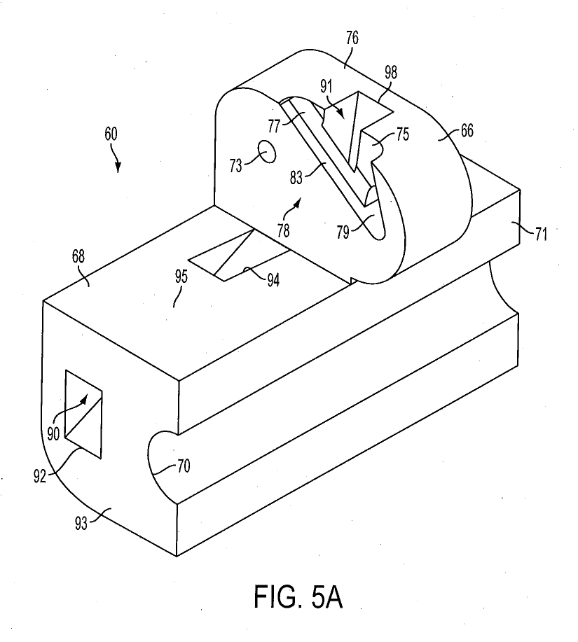

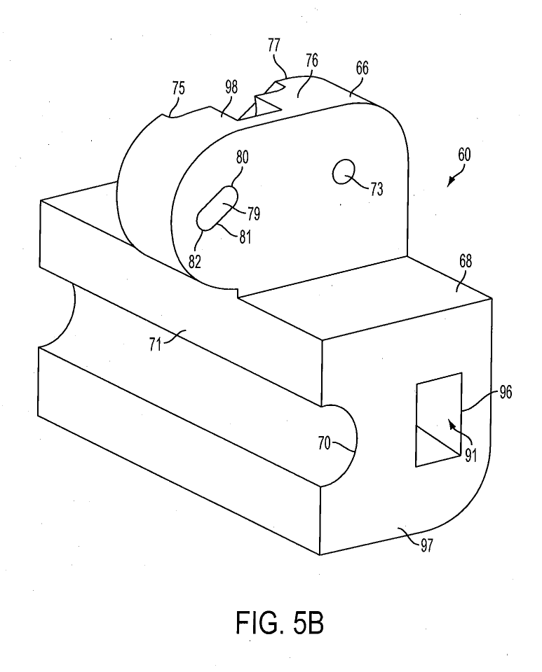

FIGS. 5A-5B depict one example of the clamp 60. The clamp 60 can include a proximal portion 66 and a distal portion 68. A longitudinal recess 70 can be formed in a confronting surface 71 of the clamp, and oriented substantially parallel with the longitudinal axis LA along the distal portion 68. The recess 70 can have a semi-circular cross-section, but other geometric cross-sections may be used so long as the recess is configured to receive at least half of the prosthesis in the compressed configuration. Each recess 70 is configured so that the recesses in the closed position together form the prosthesis retaining chamber 64 that is tubular for retaining the prosthesis 15 in the radially compressed configuration.

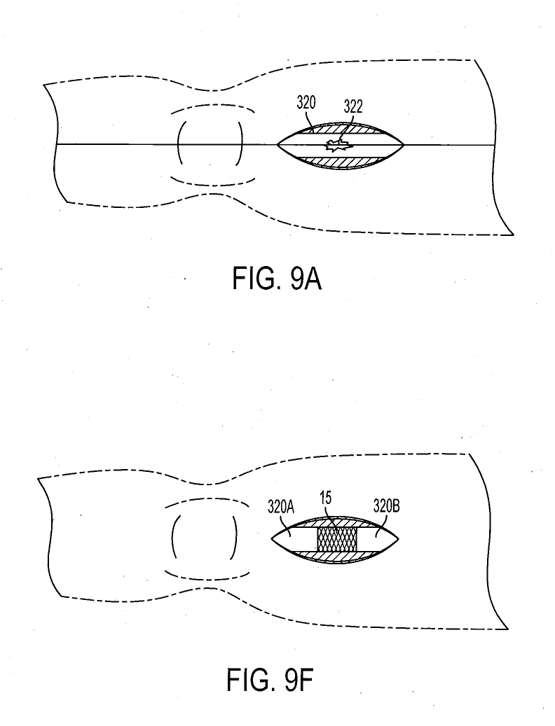

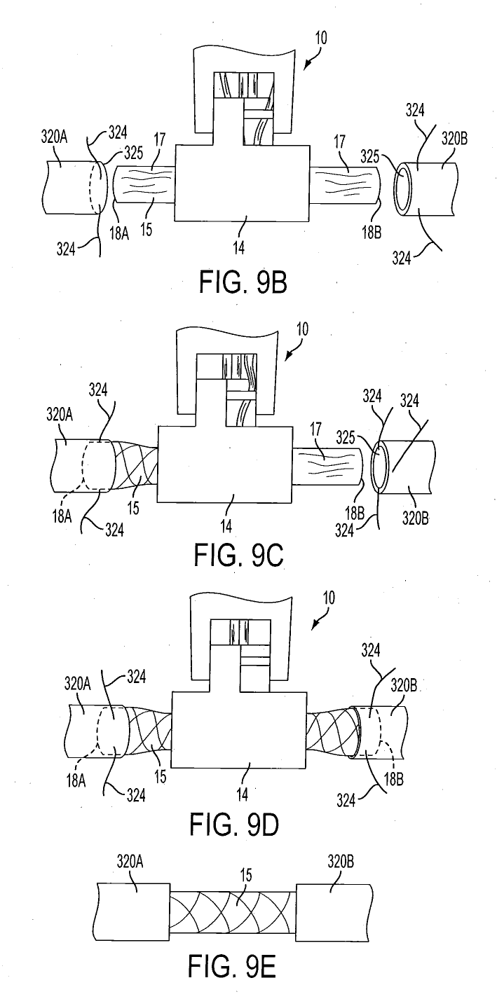

The proximal portion 66 of the clamps can protrude from the distal portion 68 in order to be received by the notch 46, where the proximal portion 66 can be coupled to the legs 48A, 48B and to the distal portion 26 of the actuation member 16. In one example, one or more pivot rods, such as pivot rods 69A, 69B, may be extended laterally across the notch 46, and be fixed in a secured position with the legs 48A, 48B. The proximal portion 66 of the clamps can have a bore 73 extending laterally through the body of proximal portion 66 for receiving the corresponding pivot rod 69A, 69B. To this end, the clamps 60, 62 can pivot about an axis PA defined by the corresponding pivot rod 69A, 69B between the closed position (FIG. 1) and the open position (FIG. 6) for delivery and deployment of the prosthesis 15 such as shown in FIG. 9E.

Further in FIGS. 5A-5B, a proximal notch 75 can be formed in the proximal surface 76 of the proximal portion 66 of the clamps. The notch 75 can be shaped to extend parallel to the translational axis TA when the clamp is in the closed configuration. An angled recess 77 can be formed in a lateral surface 78 of the proximal portion of the clamps. The notch 75 may be opened from the lateral surface 78 side, and in communication with the recess 77. The recess 77 can be shaped to extend parallel to the translational axis TA when the clamp is in the open configuration, thus the recess 77 is angled relative to the notch 75. The proximal notch 75 and the recess 77 can be shaped and sized to receive a segment of the shafts 24A, 24B adjacent the distal portion 26 thereof, as shown in FIG. 4, moving within the combined area of the notch 75 and the recess 77. A slot 79 for receiving the distal portion 26 of the actuation member 16 can be formed between the lateral surfaces of the proximal portion 66, and in communication with the notch 75 and the recess 77. The slot 79 can be shaped to extend parallel to the translational axis TA when the clamp is in the open configuration, thus the slot 79 is angled relative to the notch 75 and can be aligned with the recess 77. When the actuation member is at the first position, the distal portion 26 is positioned in the slot adjacent the first end 80 of the slot 79, such as shown in FIG. 4. Movement of the distal portion 26 within the slot 79 along the translational axis TA causes the distal portion 26 to slide against the edge 81 that defines the slot 79 toward the second end 82 of the slot 79. As a result, the clamp can pivot outwardly along the pivot axis PA and the slot is reoriented, such as substantially parallel, to the translational axis TA. The angled recess 77 provides clearance for the pivoting clamp relative to the intermediate body 24. The edge 83 of the angled recess 77 may be repositioned to lie against the intermediate body 24 when the clamp is in the open position.

According to FIGS. 4-5B, one or more bores can be formed in the clamps to allow the retraction members to pass therethrough. For example, each clamp may have two bores formed therein. For the first clamp 60, a first bore 90 receives the retraction member from the first end 18A of the prosthesis 15 and a second bore 91 receives the retraction member from the second end 18B of the prosthesis 15. Since the orientation of the second clamp 62 is opposite to the first clamp, its first bore is associated with the second end and its second bore is associated with the first end of the prosthesis. Further discussion of the clamps will be confined to the first clamp 60, with the appreciation that the same will also apply to the second clamp as appreciated by those skilled in the art.

In FIGS. 5A-5B, a first end opening 92 of the first bore 90 can be formed in a first lateral surface 93 of the distal portion 68. A second end opening 94 of the first bore 90 can be formed in a proximal surface 95 of the distal portion 68. Between the first and the second end openings 92, 94, the first bore 90 may be linear (i.e., an angled bore may extend between the end openings), L-shaped, curved-shaped, or otherwise oriented to facilitate the transmission of the retraction member from along the longitudinal axis to perpendicular along the translational axis. A first end opening 96 of the second bore 91 can be formed in a second lateral surface 97 of the distal portion 68. A second end opening 98 of the second bore 91 can be formed in the proximal surface 76 of the proximal portion 66. Between the first and the second end openings 96, 98, the second bore 91 may be linear (i.e., an angled bore may extend between the end openings), L-shaped, curved-shaped, or otherwise oriented to facilitate the transmission of the retraction member from along the longitudinal axis to perpendicular along the translational axis. In one example shown in FIG. 3, the second bore 91 is L-shaped with a first leg substantially parallel with the translational axis, and a second leg is angled within the clamp body proximally toward the first leg from the second lateral surface. The bores are configured for open communication with the channels through which the retraction member is allowed to extend. The bores can include additional features, such as bearings and/or lubrication, to facilitate translation of the retraction member therethrough.

Turning to the operation of the deployment device 10 without the removable sheath and the retraction member, FIG. 1 depicts the retaining member 14 in the closed position to retain the respective segment of the prosthesis 15 in the radially compressed configuration. The safety lock 20 is in a locking position around the button 39 to prevent any inadvertent movement of the actuation member 16 away from its first position. The biasing member may bias the actuation member in the first position. To this end, the distal portion 26 can be positioned proximate the first end 80 of the slot to urge the clamps 60, 62 of the retaining member in the closed position.

With reference to FIG. 6, an end user can first remove the safety lock from its locking position by sliding the safety lock away from the button. The biasing member can facilitate maintaining the actuation member at the first position when the safety lock is removed. The end user can then place his or her palm and fingers around the body 30 in order to place the thumb on the button 39 for its actuation. With the retaining member 14 in the closed position and the actuation member at the first position, the end user can apply a force F along the translational axis TA at the button 39, which the force is transmitted through the intermediate body 24 to the distal portion 26 of the intermediate body. Force F can be at least greater than the opposing radially inward retaining force Fs of each clamp that is provided by the biasing member.

Under the force F, the distal portion 26 of the intermediate body 24 is moved along the translational axis TA within the slot 79 of the clamps to slide against the edge 81 closer to the second end 82 of the slot 79. The confronting surfaces 71 of the clamps 60, 62 begin to separate from one another as the clamps 60, 62 begin to be pivotably displaced outward about the respective pivot axes PA. To this end, the surfaces that define the recesses 70 begin to move outward away from one another to permit the respective segment of the prosthesis 15 to expand to the expanded configuration. The slot 79 can be reoriented during movement of the clamp to a position where the slot is substantially parallel to the translational axis TA. Preferably, when the outer flange of the button contacts the proximal surface of the support frame and/or when the distal portion of the intermediate body contacts the second end of the slot, the clamps are in a position to allow for the full expansion of the prosthesis.

The components of the device can be machined or molded from a biocompatible polymer or metal as can be appreciated by those skilled in the art. Actuation member 16 can further comprise an electromechanical actuator coupled to the device, such as an electric motor coupled to a power source and/or controls for electronically controlling the speed, direction, and force of the actuation member. In one example, the actuation member can be operable with a push of a button as can be appreciated by those of ordinary skill in the art.

A concise description of prosthesis 15 will now be provided. The prosthesis can include a generally tubular graft body and/or one or more anchoring members 99 and/or supporting members together defining a fluid passageway. The prosthesis is movable between the radially compressed, delivery configuration and the radially expanded, deployed configuration. The prosthesis can be balloon expandable; however, it is preferred that the prosthesis is self-expandable. The anchoring members and/or supporting members can be attached to the graft body by sutures sewn therein, wire, staples, clips, bonding agents, or other methods that may be used to achieve a secure attachment to the graft body. The prosthesis has a size and shape suitable for at least partial placement within a body vessel, such as an artery or vein, and most particularly, for placement at the site of a vascular trauma. The prosthesis may be easily manipulated during delivery to a transected artery or vein during emergency surgery, and particularly, to obtain hemostasis while maintaining blood perfusion. The anchoring member and/or supporting member can be any stent pattern known to one skilled in the art. Examples of stent patterns is the Z-STENT® and ZILVER® stent, each available from Cook Medical Inc. (Bloomington, Ind.). The anchoring member and/or supporting member can be formed of a biocompatible metal, such as stainless steel (e.g., 316L SS), titanium, tantalum, nitinol or other shape memory materials, or a high-strength polymer. Preferably, anchoring devices can be included on at least the anchoring members to provide vessel fixation, while avoiding adverse conditions associated with disturbing the vasa vasorum and/or pressure induced necrosis of the medium muscular arteries of the type that may result from tying ligatures circumferentially around a connector or a vascular conduit. The anchoring devices can include various shaped member structures, including barbs, fibers, bristles, or outer protruding and penetrable media.

The graft body can be formed from conventional materials well known in the medical arts. The graft body may comprise an expanded polytetrafluoroethylene (ePTFE), polytetrafluoroethylene, silicone, polyurethane, polyamide (nylon), as well as other flexible biocompatible materials. The graft body can also be formed from known fabric graft materials such as woven polyester (e.g. DACRON®), polyetherurethanes such as THORALON® from Thoratec Corporation (Pleasanton, Calif.), polyethylene such as an ultra-high molecular weight polyethylene (UHMwPE), commercially available as DYNEEMA®. The graft body may also include a bioremodelable material, such as reconstituted or naturally-derived collagenous materials, extracellular matrix material (ECM), submucosa, renal capsule membrane, dermal collagen, dura mater, pericardium, fascia lata, serosa, peritoneum or basement membrane layers, intestinal submucosa, including small intestinal submucosa (SIS), stomach submucosa, urinary bladder submucosa, and uterine submucosa. One non-limiting example of a suitable remodelable material is the SURGISIS® BIODESIGN™, commercially available from Cook Medical Inc. (Bloomington, Ind.). Another suitable remodelable material is the graft prosthesis material described in U.S. Pat. No. 6,206,931 to Cook et al., which is incorporated herein by reference in its entirety.

Portions of the prosthesis can also include a coating of one or more therapeutic agents along a portion of the stent structure and/or the graft body. Therapeutic agents for use as biocompatible coatings are well known in the art. Non-limiting examples of suitable bio-active agents that may be applied to the vascular conduit include thrombo-resistant agents, antibiotic agents, anti-tumor agents, antiviral agents, anti-angiogenic agents, angiogenic agents, anti-mitotic agents, anti-inflammatory agents, angiostatin agents, endostatin agents, cell cycle regulating agents, genetic agents, including hormones such as estrogen, their homologs, derivatives, fragments, pharmaceutical salts and combinations thereof. Those skilled in the art will appreciate that other bioactive agents may be applied for a particular use. The bioactive agent can be incorporated into, or otherwise applied to, portions of the vascular conduit by any suitable method that permits adequate retention of the agent material and the effectiveness thereof for its intended purpose. Although the device has been described in connection with its primary intended use for repair of vascular trauma, those skilled in the art will appreciate that the device may also be used to repair other traumatic conditions. Non-limiting examples of such conditions include aneurysms, such as abdominal aorta aneurysms, and surgery for tumor removal.

In one example, the axial length of the prosthesis 15 relative to the length of the prosthesis retaining chamber 64 is such that the outer ends 18A, 18B of the prosthesis 15 can extend outwardly beyond the chamber ends. In this instance, the outer ends 18A, 18B of the prosthesis may be expanded slightly to a greater diameter than the radially compressed diameter of the prosthesis retained within the chamber. Preferably, such greater diameter of the outer ends of the prosthesis is less than the overall diameter of the vessel portion end opening. In one example, the prosthesis may be specially configured so that radial compression of a substantial intermediate portion of the prosthesis (e.g., at least about 80% L; L is length of prosthesis) to the first diameter, results in the outer ends of the prosthesis (e.g., each up to about 10% L) having the greater diameter that is about 30% greater than the compressed configuration. To this end, the prosthesis is structured to expand up to about 3% in diameter or less for every 1% of exposed length.

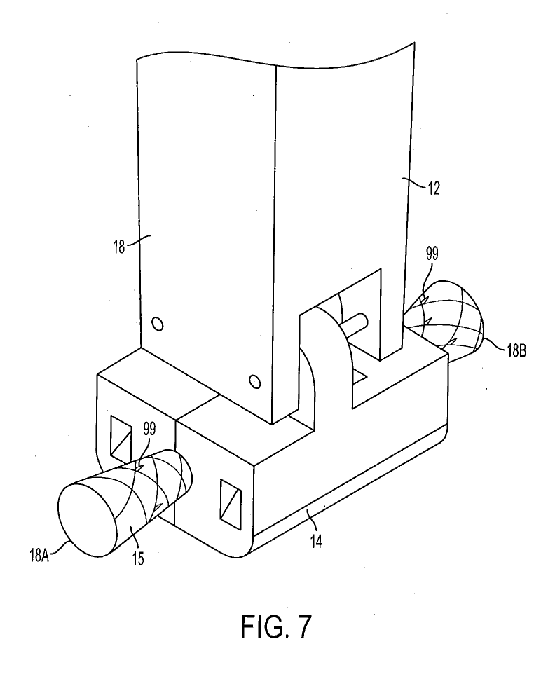

Another step of the operation may be to remove the removable sheath 17 away from the prosthesis 15 when present on the prosthesis. In FIGS. 1 and 3, the retraction members 19B, 19C are shown entering into the respective first and second bores 90, 91 formed in the clamps, where the retraction members 19B, 19C are guided to the corresponding channels 42, 44 formed in the tubular body 30. The retraction members associated with a respective outer end of the prosthesis, such as, e.g., retraction members 19A, 19B, can be coupled to one another somewhere along the channel or proximal to the channel, such as, e.g., the channel 42. This arrangement can allow for the removal of the removable sheath at one end of the prosthesis with a single retraction of both retraction members. As shown in FIG. 1, a knob 110A, 110B may be attached to the ends of the retraction members to maintain the coupling of the retraction members along each side and to improve grippabilty for the end user during retraction. To this end, the retraction members associated with the first outer end of the prosthesis and the retraction member associated with the second outer end of the prosthesis can be selectively retracted to remove the removable sheath from the outer ends of the prosthesis in sequence or simultaneously. Thus, the end user can apply a withdrawing force to the knob 110A, which is transmitted to the retractions members 19A, 19B to peel or split the removable sheath from the first outer end 18A of the prosthesis 15 to allow for expansion of the prosthesis in an outside-in direction. The end user may pull the entire retraction member and the removable sheath from the body of the patient. The same withdrawing action is performed with the knob 110B, to peel or split the removable sheath from the second outer end 18B of the prosthesis 15 to allow for expansion of the prosthesis in an outside-in direction. FIG. 7 depicts the outer ends 18A, 18B of the prosthesis 15 in the expanded configuration, while the retaining member 14 is in the closed configuration. Next, the retaining member 14 can be moved to the open configuration as previously described.

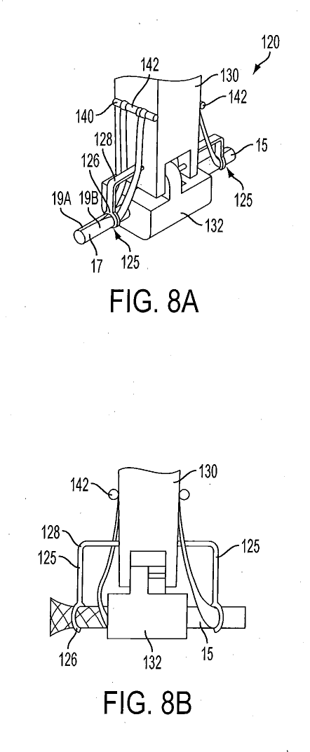

To reduce the delivery profile of the outer end of the prosthesis with the removable sheath and the retraction members, the first and second bores 90, 91 can be moved radially toward the prosthesis as close as possible, while maintaining the structural strength of the device. Another approach is shown in FIGS. 8A-8B, where another example of the deployment device 120 is provided with profile reducer mechanisms 125. The deployment device 120 can include one or more of the features described with respect to the deployment device 10. The profile reducer mechanism 125 can include a retention ring 126 and a support arm 128 to couple the retention ring 126 to the support frame 130. The retention ring 126 can have an opening so that the ring can be removed from the prosthesis. The support arm 128, which can be an L-shaped bracket, is configured to place the retention ring axially away from the retaining member 132. The support arm 128 is attached to the support frame 130 by any attachment mechanism, such as by adhesives, soldering, welding, or other common attachment mechanisms. The retention ring 126 is sized to be placed around the prosthesis 15, the retraction members 19A, 19B, and the removable sheath 17, and to provide clearance for the retraction members and the removable sheath to be moved relative to the retention ring. The retention ring 126 may also facilitate the transition between the horizontal movement of the retraction member to the vertical movement of the retraction member.

FIGS. 8A-8B show another modification to the deployment device 10, with a handling system 140 for the removable sheath once removed from the prosthesis. For example, the handling system 140 can include a spool 142 coupled to the support frame 130. The spool 142 can be attached to the end of the retraction members 19A, 19B. Instead of manually withdrawing the retraction members as described with respect to FIG. 1, the spool 142 is operative to rotate in a desired direction to wind the retraction members and the removable sheath around the spool. The spool 142 can be rotated manually or automatically by a spring loaded coil or an electric motor. In the instance of automatic rotation, controls can be associated with the spool to provide the desired direction, speed, braking, and torque for the spool. The spool 142 can be placed on a lateral surface of the support frame 130 as shown in FIGS. 8A-8B, or can be placed on the proximal end of the support frame.

FIGS. 8A-8B show an instance where the retraction members and the removable sheath are not removed from within the support frame 130. Instead, the retraction members 19A, 19B and the removable sheath 17 can be removed from outside the support frame. For example, when the handling system 140 is placed on the lateral surface of the support frame 130, the retraction members 19A, 19B and the removable sheath 17 can be guided directly to the handling system 140.

Methods of use and medical treatment with the deployment device will now be discussed. Although the discussion will focus primarily on the operation of deployment device 10, it can be appreciated that the other embodiments of the deployments devices described herein can be similarly operated and used. Although the example described below illustrates use of the deployment device to deliver a prosthesis for interconnecting a blood vessel, the deployment device can deliver a prosthesis to other lumens, ducts, and passageways of the body, and the term “body vessel” will be used to describe in general its use. It is contemplated that the device can be used to deploy a prosthesis within an opening, such as a laceration, formed in the wall of the body vessel without transecting the body vessel.

FIGS. 9A-9F illustrate a method of using the device to treat a body vessel 320. FIG. 9A depicts an example body vessel 320 found in the leg of a patient. The body vessel 320 has previously been subjected to a traumatic episode, resulting in a portion 322 of body vessel 320 being torn away or otherwise severely damaged. Pre-surgery preparation has been applied to the leg and a trauma pathway may be formed therein in order to gain access to the body vessel and the damaged portion thereof. After clamping the body vessel 320 on both ends of the portion 322 to restrict blood flow temporarily, the body vessel 320 can be cut or transected by the clinician into two portions 320A, 320B, as shown in FIG. 9B. The transection may be at the damaged portion 322 of the blood vessel 320 or as far away as necessary from the damaged portion to remove unhealthy portions of the body vessel or unrepairable portions of the body vessel. The sutures 324 can be attached to the end openings 325 of the body vessel portions 320A, 320B to keep them fixed in place and opened to facilitate insertion of the prosthesis. Forceps may also be used in a similar manner. Any number of sutures can be used to retain the end openings 325 in the open position, although triangulation sutures can be sufficient, with each suture being about 120 degrees apart from the adjacent suture. A prosthesis is selected to have a radial expanded cross-section and a longitudinal length sufficient to bridge the body vessel portions 320A, 320B and radially fit within the body vessel portions.

According to FIG. 9B, the prosthesis 15, which is preloaded within the deployment device 10, which is representative of any deployment device configurations described herein, is shown being situated and oriented adjacent the body vessel portions 320A, 320B through the trauma pathway. The prosthesis 15 is in the delivery, compressed configuration, being retained in the radially compressed configuration by the retaining member 14. The first outer end 18A of the prosthesis 15 can be inserted into the vessel portion 320A through the end opening 325 by a sufficient distance to allow the anchoring members of the prosthesis to engage within the tissue. It is preferred that the vessel portion initially selected be the non-blood supplying vessel end. The vessel portion 320A may be manually pulled over the first outer end 18A of the prosthesis 15. The removable sheath 17 can be removed from the first outer end 18A of the prosthesis, such as described herein, while inserted within the vessel portion 320A. For example, with reference to FIG. 1, the knob 100A which is coupled to the removable sheath 17 can be retracted in order to peel or split the removable sheath 17 and remove it from the first outer end 18A of the prosthesis to allow for expansion thereof. It can be appreciated by those skilled in the art that the removable sheath may be omitted from the deployment device.

In FIG. 9C, the deployment device 10 and the prosthesis 15 can then be manipulated in order to introduce the second outer end 18B of the prosthesis 15 into the vessel portion 320B through the end opening by a sufficient distance to allow the anchoring members of the prosthesis to engage within the tissue. It can be appreciated that the second outer end 18B of the prosthesis may be inserted into the vessel portion 320B prior to the removal of the removable sheath from the first outer end 18A. The vessel portion 320B may be manually pulled over the second outer end 18B of the prosthesis 15. The removable sheath 17 can be removed from the second outer end 18B of the prosthesis, such as described herein, while inserted within the vessel portion 320B to allow for expansion thereof. The vessel portions 320A, 320B can now be sealably engaged to the first and second outer ends 18A, 18B of the prosthesis 15.

In FIG. 9D, after insertion of both outer ends 18A, 18B of the prosthesis 15 into the respective portions 320A, 320B, the actuation member can be operated to pivot the clamps of the retaining member 14 away from the prosthesis 15, as described above with reference to FIG. 6. This can permit expansion of the remaining intermediate portion of the prosthesis 15. Accordingly, the prosthesis 15 is fully deployed to interconnect the first and second vessel portions 320A, 320B of the transected body vessel 320 to form a conduit for blood flow. Sutures 324 can then be removed. Preferably, portions of the exterior surfaces of the prosthesis sealably engage with the luminal walls of the body vessel to inhibit leakage of blood and to force blood to flow throughout the body vessel during emergency surgery, and particularly to obtain hemostasis of fluid stability while maintaining blood or fluid perfusion. FIG. 9E shows the prosthesis 15 deployed and interconnecting body vessel portions 320A, 320B within the leg of the patient. Prosthesis 15 can be adapted for permanent placement within the patient, thereby obviating a need for subsequent surgical intervention.

It can be appreciated by those skilled in the art that specific features of each embodiment of the deployment device are interchangeable among the device embodiments, even where no references to the specific features are made.

Drawings in the figures illustrating various embodiments are not necessarily to scale. Some drawings may have certain details magnified for emphasis, and any different numbers or proportions of parts should not be read as limiting, unless so designated in the present disclosure. Those of skill in the art will appreciate that embodiments not expressly illustrated herein may be practiced within the scope of the present invention, including those features described herein for different embodiments may be combined with each other and/or with currently-known or future-developed technologies while remaining within the scope of the claims presented here. It is therefore intended that the foregoing detailed description be regarded as illustrative rather than limiting. In addition, it should be understood that the following claims, including all equivalents, are intended to define the spirit and scope of this invention.

We claim:

1. A deployment device comprising:
a retaining member movable between a closed position and an open position, wherein in the closed position the retaining member is positioned over at least an intermediate segment of a prosthesis to retain said intermediate segment in a compressed configuration, and in the open position the retaining member is positioned away from the prosthesis to allow said intermediate segment to move to an expanded configuration; and
a sheath configured to retain at least an outer segment of the prosthesis in a compressed configuration, wherein the outer segment is associated with at least one of a first outer end and a second outer end of the prosthesis, wherein the sheath is removable from said outer segment to allow for expansion thereof when the retaining member is in the closed position.

2. The deployment device of claim 1 further comprising an actuation member coupled to the retaining member, the actuation member being movable between a first position and a second position to move the retaining member between the closed and open positions.

3. The deployment device of claim 2 further comprising a support frame to house the actuation member, and the retaining member comprises a first clamp and a second clamp pivotably coupled to the support frame.

4. The deployment device of claim 3, wherein each of the first and second clamps is slidably engaged with a distal portion of the actuation member such that movement of the actuation member between the first and second positions causes the clamps to pivot between the open and closed configurations.

5. The deployment device of claim 3 further comprising a biasing member to bias the retaining member in the closed configuration.

6. The deployment device of claim 1, wherein the sheath comprises a first retraction member coupled to a portion of the sheath associated with the first outer end of the prosthesis and a second retraction member coupled to a portion of the sheath associated with the second outer end of the prosthesis.

7. The deployment device of claim 6, wherein in response to retraction of the first and second retraction members the corresponding sheath portions are removed from the outer ends of the prosthesis to allow for expansion thereof when the retaining member is in the closed position.

8. The deployment device of claim 6, wherein the retaining member comprises a first bore and a second bore to allow for passage of the respective first and second retraction members.

9. The deployment device of claim 8 further comprising a support frame coupled to the retaining member, wherein the support frame has a first channel and a second channel formed therein to allow for passage of the respective first and second retraction members from the corresponding first and second bores.

10. The deployment device of claim 1 further comprising a profile reducer mechanism to surround the prosthesis retained by the sheath, disposed axially away from the retaining member.

11. The deployment device of claim 1 further comprising a handling system to wind the sheath when removed from the prosthesis.

12. A deployment system comprising:
a prosthesis radially movable between a compressed configuration and an expanded configuration, the prosthesis oriented about a longitudinal axis;
a retaining member movable between a closed position and an open position, wherein in the closed position the retaining member is positioned over at least an intermediate segment of the prosthesis to retain said intermediate segment in the compressed configuration, and in the open position the retaining member is positioned away from the prosthesis to allow said intermediate segment to move to the expanded configuration; and
an actuation member coupled to the retaining member and operable to move the retaining member between the closed and open positions, the actuation member being movable along a translational axis between a first position and a second position to correspond to the closed and open positions of the retaining member.

13. The deployment system of claim 12 further comprising a support frame to house the actuation member, the support frame having a proximal end and a distal end, wherein the actuation member is operable from the proximal end of the support frame, and the retainer member is pivotably coupled to the distal end of the support frame.

14. The deployment system of claim 13 further comprising a safety lock removable engaged with the actuation member, wherein when engaged into a locking position the safety lock maintains the actuation member in the first position.

15. The deployment system of claim 12 further comprising a sheath configured to retain at least an outer segment of the prosthesis in the compressed configuration, wherein the outer segment is associated with at least one of a first outer end and a second outer end of the prosthesis, wherein the sheath is removable from said outer segment to allow for expansion thereof when the retaining member is in the closed position.

16. The deployment system of claim 15 further comprising a first retraction member coupled to a portion of the removable sheath associated with the first outer end of the prosthesis and a second retraction member coupled to a portion of the removable sheath associated with the second outer end of the prosthesis, wherein in response to retraction of the first and second retraction members the corresponding removable sheath portions are removed from the outer ends of the prosthesis to allow for expansion thereof.

17. The deployment system of claim 12, wherein the retaining member comprises a first clamp and a second clamp coupled to a distal end of the actuation member, each of the clamps configured to pivot about a pivot axis in response to linear movement of the distal end of the actuation member along the translational axis that is substantially perpendicular to the longitudinal axis.

18. A method of treating a body vessel, comprising:
inserting a first outer end of a prosthesis in a first vessel portion of a body vessel, wherein the prosthesis has an intermediate segment retained in a compressed configuration with a retaining member;
inserting a second outer end of the prosthesis in a second vessel portion of the body vessel; and
removing the retaining member from the intermediate segment of the prosthesis, whereby the intermediate segment and the first and second outer ends of the prosthesis are allowed to move to the expanded configuration.

19. The method of claim 18, wherein the first and second outer ends of the prosthesis are retained in the compressed configuration with a retaining sheath, the method further comprising the step of removing the retaining sheath from the first and second outer ends after the inserting steps to allow for expansion and engagement between the first and second outer ends of the prosthesis and the corresponding first and second vessel portions prior to the removing the retaining member step.

20. The method of claim 18, wherein the intermediate segment of the prosthesis is retained in the compressed configuration by a first clamp and a second clamp, wherein an actuation member is coupled to the first and second clamps and is operable to cause movement of the clamps away from intermediate segment to allow for expansion of the intermediate segment of the prosthesis.
